SEQUENCE LISTING (29 sequences)

SEQ ID NO: 1 - DNA, 24 bp, Artificial Sequence
  Synthetic oligonucleotide
ctcctctgcatctagccccttcca

SEQ ID NO: 2 - DNA, 48 bp, Artificial Sequence
  Synthetic oligonucleotide
gttgttgttgttatggtctaatttaaatatggtctaaagaagaagaat

SEQ ID NO: 3 - DNA, 96 bp, Artificial Sequence
  Synthetic oligonucleotide
ctcctctgcatctagccccttccagttgttgttgttatggtctaatttaaatatggtctaaagaagaagaatctcctctgcatctagccccttcca

SEQ ID NO: 4 - DNA, 19 bp, Artificial Sequence
  Synthetic oligonucleotide
cagtggaaggggcatgcag

SEQ ID NO: 5 - DNA, 21 bp, Artificial Sequence
  Synthetic oligonucleotide
tggaaggggcatgcagaggag

SEQ ID NO: 6 - DNA, 21 bp, Artificial Sequence
  Synthetic oligonucleotide
ctcctctgcatgccccttcca

SEQ ID NO: 7 - DNA, 26 bp, Artificial Sequence
  Synthetic oligonucleotide
tgtatcgttccaattttatcggatgt

SEQ ID NO: 8 - DNA, 100 bp, Artificial Sequence
  Synthetic oligonucleotide
caccctcctctgcatctagccccttccagttgttgttgttatggtctaatttaaatatggtctaaagaagaagaatctcctctgcatctagccccttcca

SEQ ID NO: 9 - DNA, 100 bp, Artificial Sequence
  Synthetic oligonucleotide
tggaaggggctagatgcagaggagattcttcttctttagaccatatttaaattagaccataacaacaacaactggaaggggctagatgcagaggagggtg

SEQ ID NO: 10 - DNA, 20 bp, Artificial Sequence
  Synthetic oligonucleotide
gtacaaaaagcaggctccgc

SEQ ID NO: 11 - DNA, 20 bp, Artificial Sequence
  Synthetic oligonucleotide
agatgcagaggagattcttc

SEQ ID NO: 12 - DNA, 20 bp, Artificial Sequence
  Synthetic oligonucleotide
caagatttggagatcccgtg

SEQ ID NO: 13 - DNA, 19 bp, Artificial Sequence
  Synthetic oligonucleotide
aatgctccacggcgaacag

SEQ ID NO: 14 - DNA, 42 bp, Artificial Sequence
  Synthetic oligonucleotide
cccctttcgccaggggtaccgtaattcatccaggtctccaag

SEQ ID NO: 15 - DNA, 43 bp, Artificial Sequence
  Synthetic oligonucleotide
ctgcatgccccttccactgtgccacggatcatctgcacaactc

SEQ ID NO: 16 - DNA, 46 bp, Artificial Sequence
  Synthetic oligonucleotide
acagtggaaggggcatgcaggttttagagctagaaatagcaagtta

SEQ ID NO: 17 - DNA, 42 bp, Artificial Sequence
  Synthetic oligonucleotide
tacgaattcgagctcggtaccgctgtgccgtacgacggtacg

SEQ ID NO: 18 - DNA, 19 bp, Artificial Sequence
  Synthetic oligonucleotide
ctgaaacccgctttgcgag

SEQ ID NO: 19 - DNA, 20 bp, Artificial Sequence
  Synthetic oligonucleotide
gggagggcaaaatcgggata

SEQ ID NO: 20 - DNA, 20 bp, Artificial Sequence
  Synthetic oligonucleotide
tacaatgccgtagcagaggt

SEQ ID NO: 21 - DNA, 20 bp, Artificial Sequence
  Synthetic oligonucleotide
cagtacactcgggaactttg

SEQ ID NO: 22 - DNA, 20 bp, Artificial Sequence
  Synthetic oligonucleotide
agatcacctaaccgatggcg

SEQ ID NO: 23 - DNA, 20 bp, Artificial Sequence
  Synthetic oligonucleotide
gcagttactggagcatcaga

SEQ ID NO: 24 - DNA, 20 bp, Artificial Sequence
  Synthetic oligonucleotide
cttgctgtcgtggtttctcg

SEQ ID NO: 25 - DNA, 20 bp, Artificial Sequence
  Synthetic oligonucleotide
cgccagtacactctgaaacc

SEQ ID NO: 26 - DNA, 20 bp, Artificial Sequence
  Synthetic oligonucleotide
ggccgaagtagtacaagcca

SEQ ID NO: 27 - DNA, 20 bp, Artificial Sequence
  Synthetic oligonucleotide
gccatgaacttacagcacgc

SEQ ID NO: 28 - DNA, 20 bp, Artificial Sequence
  Synthetic oligonucleotide
tcgacgaggccgaagtagta

SEQ ID NO: 29 - DNA, 20 bp, Artificial Sequence
  Synthetic oligonucleotide
tacagcgcggacagagattc

(19) United States
(12) Patent Application Publication
(10) Pub. No.: US 2025/0297273 A1
(43) Pub. Date: Sep. 25, 2025

(54) USE OF MIR528 IN PRODUCTION AND BREEDING OF GRAMINEOUS FORAGE GRASSES

(71) Applicant: INSTITUTE OF GENETICS AND DEVELOPMENTAL BIOLOGY, CHINESE ACADEMY OF SCIENCES, Beijing (CN)

(72) Inventors: Xiao Feng CAO, Beijing (CN); Rong Xin YANG, Beijing (CN); Xian Wei SONG, Beijing (CN); Shan Jie TANG, Beijing (CN); Qing Hua ZHAO, Beijing (CN)

(73) Assignee: INSTITUTE OF GENETICS AND DEVELOPMENTAL BIOLOGY, CHINESE ACADEMY OF SCIENCES, Beijing (CN)

(21) Appl. No.: 18/860,889

(22) PCT Filed: Apr. 9, 2022
(86) PCT No.: PCT/CN2022/090135

§ 371 Date: Oct. 28, 2024

(51) Int. Cl. C12N 15/82; C12N 9/22; C12N 15/11

(52) U.S. Cl. CPC C12N 15/8262; C12N 9/226; C12N 15/111; C12N 15/8205; C12N 2310/20

(57) ABSTRACT

Disclosed herein is use of miR528 in production and breeding of gramineous forage grasses. By down-regulating the expression and/or function of miR528, the present invention can significantly enhance the tillering and/or regeneration capacity of the gramineous forage grasses, which is conducive to the biomass accumulation of the gramineous forage grasses, and the breeding of new gramineous forage grass subspecies with enhanced tillering and/or regeneration capacity.

### TECHNICAL FIELD

The application relates to the use of miR528 in the production and breeding of gramineous forage grasses. Specifically, the application relates to application of down-regulation of miR528 expression/function in improving the yield, tillering capacity and regeneration capacity of gramineous forage grasses.

### BACKGROUND OF THE INVENTION

Grasslands are an essential component of ecological agriculture, playing a crucial role in the sustainable development of modern agriculture and animal husbandry as well as the ecological environment for human survival. However, due to long-term overexploitation and predatory utilization, the area of “three types of transformations” (degradation, desertification, and salinization) of grasslands has been increasing day by day. This has led to escalating conflicts between grasslands and livestock, significant water and soil loss, and progressive deterioration of the ecological environment. Consequently, this situation has had a severe impact on development of economy, ecology, and various social undertakings. For this reason, grassland ecological protection and restoration work is imperative. Comprehensive improvement and selection of high-yielding, high-quality forage species is one of the most effective ways to protect and restore grassland ecology.

Gramineous forage grasses are a significant category of forage grasses that typically have advantages of high grass quality, good palatability, and rich nutrition, etc. In addition to serving as high-quality forage, some gramineous grass species can also be used to produce bioenergy. Panicum virgatum is native to North America and possesses the characteristics of such as fast growth, high yield, and strong environmental adaptability. In particular, its lignocellulose content is extremely high, and its ethanol conversion rate can reach 57%. Therefore, the application of Panicum virgatum in bioenergy has garnered widespread attention in the United States.

Leymus chinensis is widely distributed in Eurasia steppe and is one of the important forage grasses in natural pastures. Leymus chinensis is also known as the “King of Gramineous Grasses” due to its exceptional qualities as well as cold resistance, drought resistance, and alkali resistance. Leymus chinensis serves not only as forage but also as a beneficial plant for grassland restoration and for soil and water conservation because its rhizomes possess strong penetration and expansion capabilities, enabling the formation of a well-developed underground rhizome network which plays a crucial role in entangling and stabilizing the soil.

Tillering, a unique branching characteristic that occurs at the basal node of the stem independent of the main stem during the growth and development of gramineous forage grasses, is one of the most crucial agronomic traits of gramineous forage grasses which directly impacts the yield of grass and seed production. The regeneration capacity is the primary characteristic of gramineous forage grasses, directly influencing the yield and mowing characteristics of the forage grasses. Cattle and sheep browsing or manual mowing during grazing can remove the meristem at the top of the plant, promoting the germination or elongation of recessive tillers and buds at the base, thereby stimulating regeneration of forage grasses. Breeding gramineous forage grasses with strong tillering and regeneration capacity is one of the crucial methods to fundamentally address the current contradiction between grass and livestock, restore and safeguard grassland resources, and preserve the green ecological environment.

### SUMMARY OF THE INVENTION

Through extensive research, the inventors of this application found that miR528 plays a crucial regulatory role in the tillering and regeneration capacity of gramineous forage grasses, such as Panicum virgatum and Leymus chinensis. That is, down-regulation of the expression and/or function of miR528 in gramineous forage grasses can significantly increase the tiller number and/or regeneration capacity of gramineous forage grasses.

Therefore, the invention provides a method for improving the tillering and/or regeneration capacity of gramineous forage grasses, which comprises down-regulating the expression and/or function of miR528 in gramineous forage grasses.

In some embodiments, the gramineous forage grasses comprise, but are not limited to, Panicum virgatum, Leymus chinensis, Brachypodium distachyon, Miscanthus, Elytrigia intermedia, Uraria crinita, Setaria viridis, Anastatica, Elymus sibiricus, Panicum miliaceum, and Bromus inermis. In some embodiments, the gramineous forage grasses are Panicum virgatum and Leymus chinensis.

In some embodiments, down-regulation of the expression of miR528 is achieved by gene knockout or gene editing or the like technologies of the coding gene of miR528, MIR528. In some embodiments, down-regulation of the expression of miR528 is achieved through gene editing of the transcribed region of the coding gene MIR528, and/or its upstream regulatory regions, and/or its downstream regulatory regions. In some embodiments, CRISPR/Cas9 system is used to perform gene editing on the transcribed region of the coding gene MIR528, and/or its upstream regulatory regions, and/or its downstream regulatory regions. In some embodiments, an sgRNA that is reverse complementary to the coding gene MIR528 is used in the CRISPR/Cas9 system: CAGTGGAAGGGGCATGCAG (SEQ ID NO: 4), wherein the Cas9 protein is targeted to the coding gene MIR528 to modify the sequence of the coding gene with the said CRISPR/Cas9 system, resulting in down-regulation of miR528 expression.

In some embodiments, the function of miR528 is down-regulated by a ST™ method. In some embodiments, the functional down-regulation of miR528 is performed by expressing a sequence that can be recognized by and interact with miR528 in gramineous forage grasses. In some embodiments, the functional down-regulation of miR528 is performed by expressing an artificial target mimic sequence or a short tandem target mimic, that is recognized by and interacts with miR528 without being cleaved by miR528, in gramineous forage grasses. In some embodiments, the function of miR528 is down-regulated by introducing a vector that expresses an artificial target mimic sequence and/or a short tandem target mimic into gramineous forage grasses, wherein the vector constitutively expresses the artificial target mimic sequence or the short tandem target mimic. In some embodiments, the artificially-synthesized target mimic sequence comprises or consists of the nucleotide sequence shown in SEQ ID NO: 1. In some embodiments, the short tandem target mimic is a sequence (SEQ ID NO: 3) obtained by linking two artificially-synthesized target mimic sequences (SEQ ID NO: 1) via a spacer sequence (SEQ ID NO: 2) of 48 nt, which exerts the effect of downregulating the function of miR528.

The invention also provides the application of down-regulation of miR528 expression/function in improving the tillering and regeneration capacity of gramineous forage grasses.

The invention also provides an artificial target mimic sequence and a short tandem target mimic (STTM) that can achieve the functional down-regulation of miR528 in gramineous forage grasses. In some embodiments, the artificially synthesized target mimic sequence comprises or consists of the nucleotide sequence set forth in SEQ ID NO: 1. In some embodiments, the short tandem target mimic comprises or consists of the nucleotide sequence set forth in SEQ ID NO: 3.

The present invention also provides a CRISPR-Cas9 vector and its corresponding sgRNA which are capable of knocking out the coding gene for miR528, MIR528, or performing gene editing on the gene in gramineous forage grasses.

The inventors of the invention found that down-regulating the expression and/or function of miR528 has no adverse effect on the plant height of gramineous forage grasses, and thus can greatly increase the biomass accumulation of gramineous forage grasses in addition to significantly enhancing the tillering of gramineous forage grasses. In addition, down-regulation of the expression and/or function of miR528 enhances the regeneration capacity of gramineous forage grasses and contributes to an increase in the total amount of forage grasses. In terms of gramineous forage grasses, which can serve as both forage grasses and bioenergy plants, an increase in total supply is beneficial for the development of livestock farming and the ethanol fuel industry.

Those skilled in the art can utilize the above findings to improve and breed new forage grass lines with down-regulated expression and/or function of miR528 to facilitate the promotion of this technology. Therefore, the invention also provides a method for breeding a new line of gramineous forage grass with enhanced tillering and/or regeneration capacity, which involves down-regulating the expression and/or function of miR528 in the new line of gramineous forage grass.

### DESCRIPTION OF DRAWINGS

The content of the application will be further illustrated below with reference to the attached drawings, which are only illustrative and do not limit the scope of the invention.

FIG. 1 illustrates the alignment results of miR528 precursor sequences in various gramineous forage grasses that have been sequenced.

FIG. 2 illustrates that the mature sequence of miR528 is present in various gramineous forage grasses.

FIG. 3 illustrates the STTM sequence used to inhibit the function of miR528 in Example 3. The STTM sequence contains two miR528 binding sites, each of which has a “bulge” of 3 bases (CTA) in the middle, so that the STTM sequence cannot be cleaved by miR528.

FIG. 4 illustrates the STTM expression vector constructed in Example 4, in which the STTM sequence that specifically binds to miR528 is driven by a constitutive promoter, the Ubi promoter.

FIG. 5 illustrates the STTM expression level in Panicum virgatum introduced with the STTM expression vector in Example 5.

FIG. 6 illustrates the phenotype of Panicum virgatum introduced with the STTM expression vector in Example 6, wherein the Panicum virgatum introduced with the STTM expression vector exhibits an increase in tiller number and biomass compared to the Panicum virgatum introduced with the empty vector.

FIG. 7 illustrates the regeneration phenotype of the Panicum virgatum introduced with the STTM expression vector 10 days after mowing in Example 7, wherein the regeneration capacity of the Panicum virgatum introduced with the STTM expression vector is enhanced after mowing, compared to the Panicum virgatum introduced with the empty vector.

FIG. 8 illustrates the identification results for sequence editing of miR528 in Panicum virgatum after introduced with the miR528 CRISPR/Cas9 vector in Example 9.

FIG. 9 illustrates the identification results for sequence editing of miR528 in Leymus chinensis after introduced with the miR528 CRISPR/Cas9 vector in Example 9.

FIG. 10 illustrates that the transgenic miR528-knockout Panicum virgatum in Example 10 exhibits a phenotype of enhanced regeneration capacity.

FIG. 11 illustrates that the transgenic miR528-knockout Leymus chinensis in Example 10 exhibits a phenotype of enhanced regeneration capacity.

### DETAILED DESCRIPTION OF THE INVENTION

The content of the application will be further illustrated below with reference to the description of specific embodiments, which are only for exemplary illustration, and do not limit the scope of the invention. Modifications, replacements and omissions may be made to each element in the embodiments of the application, without departing from the essence of the invention. The scope of protection of this application is determined by the claims and their equivalents.

Herein, the term “miRNAs” refers to a class of endogenous small microRNAs with a length of 21-24 nt, which do not encode proteins but instead negatively regulate the expression of target genes at the post-transcriptional level by complementary base pairing, and thereby participate in processes such as growth, development, and environmental responses in organisms. In plants, miRNAs mainly negatively regulate the expression of target genes by degrading target mRNAs, thereby exerting regulatory functions in a variety of biological processes.

Herein, the term “Short Tandem Target Mimic” or “STTM” refers to a method for inhibiting the function of a miRNA. Specifically, STTM consists of two artificially synthesized miRNA target mimic sequences connected in series, with a 3-base bulge structure provided at the miRNA cleavage site in each of the two target mimics. The structure enables the miRNA to bind to the target mimic but prevents the miRNA from cleaving it. As a result, the target mimic binding can adsorb miRNA and inhibit its regulation of genuine endogenous target genes.

Herein, the term “CRISPR-Cas9 (Clustered Regularly Interspaced Short Palindromic repeats/CRISPR associated 9) system” refers to a gene editing system. Because of its accuracy and ease of use, the CRISPR-Cas9 system has gradually replaced traditional genome editing technologies such as Zinc Finger Nuclease (ZFN) and Transcription Activator-like Effector Nucleases (TALEN), and has been widely utilized in the fields of gene therapy and crop improvement. The principle is that the endonuclease Cas9 cuts a double-stranded DNA at a target site under the targeting effect of guide RNA (sgRNA), and mutations are introduced in the broken DNA during the repair process to achieve sequence modification in the target gene. Previous studies have shown that the miRNA coding region can be targeted for editing to inhibit the function of the miRNA using CRISPR/Cas9 technology.

Herein, the term “miR528” refers to one of the earliest reported miRNA family members in rice which is conserved in monocots. Chinese patent application CN201410423077.X describes the function of miR528 in heading period and virus resistance of rice. Specifically, overexpression of miR528 advances the heading time of rice, while down-regulation of expression enhances rice's resistance to RSV virus and delays the heading time of rice.

Through extensive research, the inventors of this application found that miR528 plays a crucial regulatory role in the tillering and regeneration capacity of gramineous forage grasses, such as Panicum virgatum and Leymus chinensis. That is to say, down-regulation of the expression and/or function of miR528 in gramineous forage grasses can significantly increase the tiller number and/or regeneration capacity of gramineous forage grasses. Therefore, the invention provides the use of miR528 in the production and breeding of gramineous forage grasses.

(1) Application of miR528 in the Production of Gramineous Forage Grasses

A first aspect of the invention provides a method for enhancing the tillering and/or regeneration capacity of gramineous forage grasses, thereby increasing the biomass accumulation of gramineous forage grasses and contributing to a rise in the total amount of forage grasses. In terms of gramineous forage grasses, which can serve as both forage grasses and bioenergy plants, an increase in total supply is beneficial to the development of livestock farming and the ethanol fuel industry. Said method includes down-regulating the expression and/or function of miR528 in gramineous forage grasses.

In some embodiments, the gramineous forage grasses may include, but are not limited to, Panicum virgatum, Leymus chinensis, Brachypodium distachyon, Miscanthus, Elytrigia intermedia, Uraria crinita, Setaria viridis, Anastatica, Elymus sibiricus, Panicum miliaceum, and Bromus inermis. In some embodiments, the gramineous forage grasses are Panicum virgatum and Leymus chinensis.

In some embodiments, down-regulation of the expression of miR528 can be achieved by gene knockout or gene editing of the gene encoding miR528, MIR528.

In some embodiments, down-regulation of the expression of miR528 can be achieved by gene editing of the transcribed region of the coding gene MIR528, and/or its upstream regulatory regions, and/or its downstream regulatory regions.

In some embodiments, the CRISPR/Cas9 system can be used to perform gene editing on the transcribed region of the coding gene MIR528, and/or its upstream regulatory regions, and/or its downstream regulatory regions. In some embodiments, an sgRNA, CAGTGGAAGGGGCATGCAG (SEQ ID NO: 4), that is reverse complementary to the coding gene MIR528, is used in the CRISPR/Cas9 system. The Cas9 protein is targeted to the coding gene MIR528 to induce a modification (including base deletion, substitution, or insertion) in the sequence of the coding gene using the said CRISPR/Cas9 system, which results in down-regulation of miR528 expression.

In some embodiments, the function of miR528 can be down-regulated by the STTM method. In some embodiments, the STTM method may comprise expressing a sequence that is recognizable by and interacts with miR528 in gramineous forage grasses. In some embodiments, the STTM method may comprise expressing an artificially synthesized target mimic sequence or a short tandem target mimic that is recognizable by and interacts with miR528 without being cleaved by miR528 in gramineous forage grasses. In some embodiments, the STTM method may comprise introducing a vector that expresses an artificially synthesized target mimic sequence and/or a short tandem target mimic into gramineous forage grasses to down-regulate the function of miR528, wherein the vector constitutively expresses the artificially synthesized target mimic sequence or the short tandem target mimic.

In some embodiments, the artificially synthesized target mimic sequence may comprise or consist of the following nucleotide sequence:

(SEQ ID NO: 1)
CTCCTCTGCATCTAGCCCCTTCCA.

In some embodiments, a short tandem target mimic that can also down-regulate the function of miR528 is obtained from two artificially synthesized target mimic sequences in series separated by a 48 nt spacer sequence, wherein the spacer sequence is as follows: gttgttgttgttatggtctaatttaaatatggtctaaagaagaagaat (SEQ ID NO: 2). The final functional short tandem target mimic comprises or consists of the nucleotide sequence:

(SEQ ID NO: 3)
CTCCTCTGCATCTAGCCCCTTCCAgttgttgttgt
tatggtctaatttaaatatggtctaaagaagaaga
atCTCCTCTGCATCTAGCCCCTTCCA.

(2) Application of miR528 in the Breeding of Gramineous Forage Grasses

A second aspect of the invention also provides a method for breeding a new line of gramineous forage grass with enhanced tillering and/or regeneration capacity, comprising down-regulating the expression and/or function of miR528 in the said new line of gramineous forage grass, e.g., compared to that in a parent gramineous forage grass or a wild-type gramineous forage grass.

In some embodiments, the method for down-regulating the expression and/or function of miR528 in the new line of gramineous forage grass may be as described in part (1) above.

(3) CRISPR-Cas9 Vector Used for Achieving Knock-Out or Gene Editing of the Coding Gene for miR528 (MIR528) in Gramineous Forage Grasses, and the sgRNA Thereof

A third aspect of the invention also provides a CRISPR-Cas9 vector capable of achieving knock-out or gene editing of the coding gene for miR528 (MIR528) in gramineous forage grasses, and sgRNA thereof. In some embodiments, the sgRNA comprises or consists of the nucleotide sequence: CAGTGGAAGGGGCATGCAG (SEQ ID NO: 4). By using the CRISPR-Cas9 vector, the Cas9 protein can be targeted to the miR528-coding gene (MIR528) to edit the coding gene, such as to produce deletion, insertion or mutation of bases, etc., leading to a decrease in the abundance of miR528 expression.

### (4) Artificially Synthesized Target Mimic Sequences and Short Tandem Target Mimics (STTM)

A fourth aspect of the invention also provides an artificially synthesized target mimic sequence and a short tandem target mimic (STTM) capable of achieving the functional down-regulation of miR528 in gramineous forage grasses, both of which can be used in the STTM method to achieve the functional down-regulation of miR528 in gramineous forage grasses.

In some embodiments, the artificially synthesized target mimic sequence comprises or consists of the nucleotide sequence set forth in SEQ ID NO: 1. In some embodiments, the short tandem target mimic comprises or consists of the nucleotide sequence set forth in SEQ ID NO:3.

The content of the invention is described in detail below with specific examples.

### Example 1. BLAST and Sequence Alignment of the miR528 Precursor

Using the coding sequence for the mature region of rice miR528: TGGAAGGGGCATGCAGAGGAG (SEQ ID NO:5), a BLAST was conducted on plant species with sequenced genomes available in Phytozome V13 (https://phytozome-next.jgi.doe.gov/). Folding analysis of secondary sequences was performed on completely matched sequences and their flanking sequences (200 bp on each side), and the sequences capable of folding into stem-loop structures were extracted and saved in FASTA format. Alignment and editing of sequences were conducted using Clustal X2 and Genedoc.

The results of the alignment are shown in FIG. 1. The results showed that miR528 precursors were present in the genomes of various gramineous forage grasses.

### Example 2. Detection of Mature miR528 in Various Forage Grasses

A DNA sequence, CTCCTCTGCATGCCCCTTCCA (SEQ ID NO: 6), that is complementary to the mature sequence of miR528, and a DNA sequence, TGTATCGTTCCAATTTTATCGGATGT (SEQ ID NO: 7), that is complementary to the U6 snRNA sequence, were artificially synthesized to serve as internal controls for loading amounts among different materials. The DNA sequences above were isotopically end-labelled with γ-³²P and polynucleotide kinase T4 PNK (NEB, M0201S), respectively (the labeling system for probes is shown below), and these labeled DNA sequences were used as hybridization probes.

The system for T4 PNK labeling of probes included: 1 μl of (10 μM) DNA probes, 5 μl of 10× T4 kinase Buffer, 5 μl of γ-³²P-labeled ATP, 1 μl of T4 PNK (10U/μl), and 38 μl of ultrapure water. The mixture was incubated on a heating block at 37° C. for 1 hour.

Total RNA was extracted from various forage grasses materials using TRNzol (TIANGEN), electrophoresed in a 15% polyacrylamide gel containing 7M urea at a voltage of 200V for 2 hours, and then transferred to a membrane for hybridization. The hybridization reaction was carried out overnight at 42° C., and the membrane was washed twice with membrane washing buffer (2×SSC, 0.2% SDS), and then wrapped in plastic wrap. The isotope signal was collected using a Typhoon FLA9500 laser scanning imager, and the accumulation of miR528 was characterized based on the signal intensity.

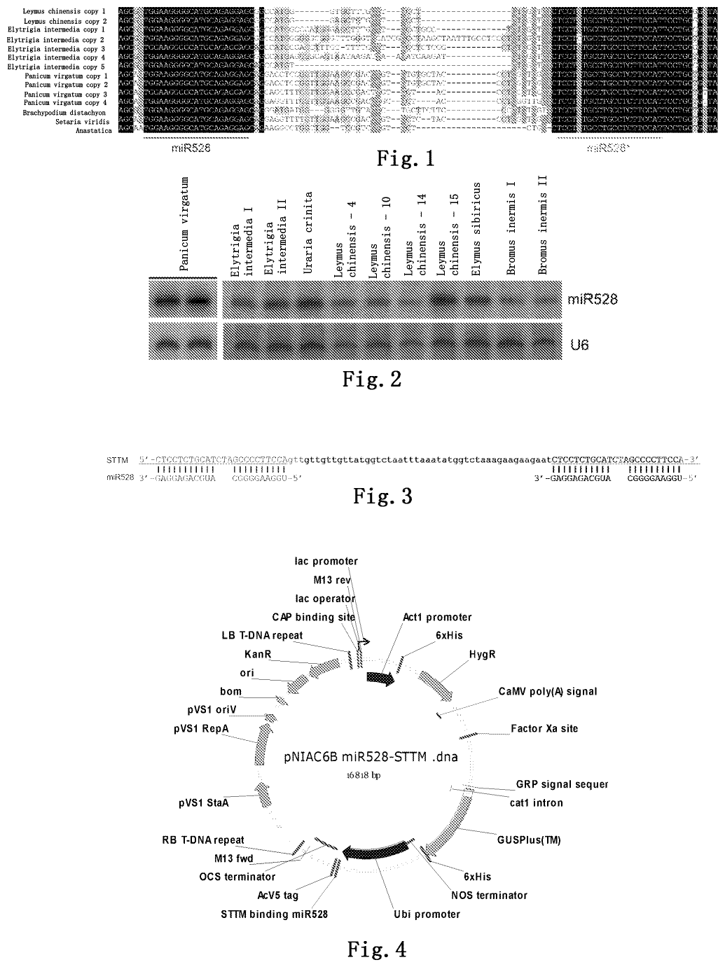

The hybridization results are shown in FIG. 2, indicating that mature miR528 could be detected in various forage grasses as tested.

### Example 3. Design of a Mature miR528-Specific STTM Sequence

A STTM sequence specific to mature miR528 was designed, as illustrated in FIG. 3. Specifically, a miR528 binding site was designed at both ends of a sequence with a total length of 96 bases, and 3 bases were inserted between the 10th and 11th bases of the miR528 binding region.

The specific sequence shown in SEQ ID NO: 3 can bind to miR528 but will not be cleaved by post-transcriptional regulation mediated by miR528.

### Example 4. Construction of miR528 STTM Transgenic Vector

First, the DNA sequence CF3380 (SEQ ID NO:8) and its reverse complementary sequence CF3381 (SEQ ID NO:9) were synthesized.

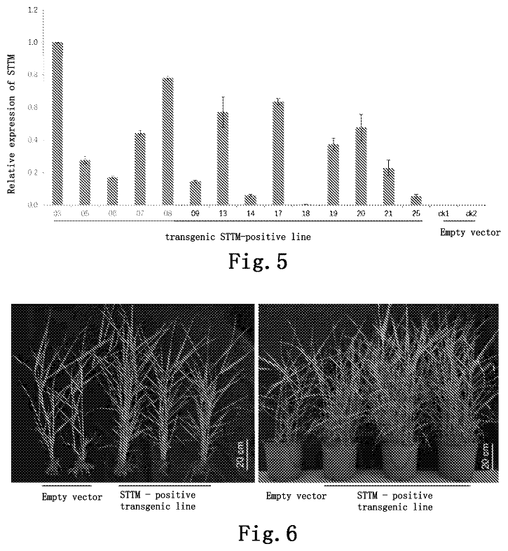

CF3380 and CF3381 were mixed in equal moles, denatured at 95° C. for 10 minutes, then naturally cooled to room temperature, and annealed to form a double-stranded DNA, which was subsequently recombined into an intermediate vector using the pENTR™/TEV/D-TOPO™ Cloning Kit (ThermoFisher, K253520) to obtain a DNA fragment of interest; the DNA fragment of interest was then inserted into a binary expression vector, pNIAC6B, allowing it to be positioned downstream of the strong maize Ubquintin promoter, using the Gateway™ LR Clonase™ Kit (ThermoFisher, 11791020). Specific experimental procedures were conducted in accordance with the instructions of the Kit, and the final binary expression vector map obtained is shown in FIG. 4.

### Example 5. Identification of miR528 STTM Transgenic Lines

Using the agrobacterium-mediated callus infection system, the STTM transgenic vector constructed in Example 4 was transferred into Panicum virgatum callus. Additionally, a portion of the callus transferred with an empty vector was used as a control. After differentiation and emergence, the callus was transferred to vermiculite nutrient soil for cultivation. After 4 months, TRNzol (TIANGEN) was used to extract the total RNA of transgenic Panicum virgatum. 2 μg of total RNA was taken from each sample, and cDNA was generated by reverse transcription using HiScript II 1st Strand CDNA Synthesis Kit (Vazyme, R211). Subsequently, quantitative PCR was employed to detect the expression level of STTM RNA in each transgenic line. The primers CF6738 (SEQ ID NO:10) and CF6739 (SEQ ID NO:11) were used to detect the STTM expression, while the primers CF6095 (SEQ ID NO:12) and CF6096 (SEQ ID NO:13) were used to amplify the internal control gene ACTIN.

The STTM expression levels in each transgenic line were shown in FIG. 5. High levels of STTM RNA expression were detected in various independent transgenic lines, while no STTM RNA expression was detected in the transgenic lines transformed with the empty vector.

### Example 6. Identification of Tiller Number of miR528 STTM Transgenic Panicum virgatum

The phenotype of 4-month-old transgenic Panicum virgatum grown in vermiculite nutrient soil was checked. As shown in FIG. 6, compared with the control line transformed with the empty vector, the transgenic in which miR528 was inhibited exhibited more branches and an increase in total biomass with growth height not affected. In other words, the transgenic lines with inhibited miR528 showed stronger tillering capacity and greater biomass accumulation.

### Example 7. Identification of Regeneration Capacity of miR528 STTM Transgenic Panicum virgatum after Mowing

The top parts of 4-month-old transgenic Panicum virgatum seedlings, grown in vermiculite nutrient soil, were uniformly mowed, leaving only 15 cm-long stems above ground. Each plant was then cut into equal sections from the roots, replanted and cultured in the vermiculite nutrient soil, and the phenotype was checked for new tillers after 10 days.

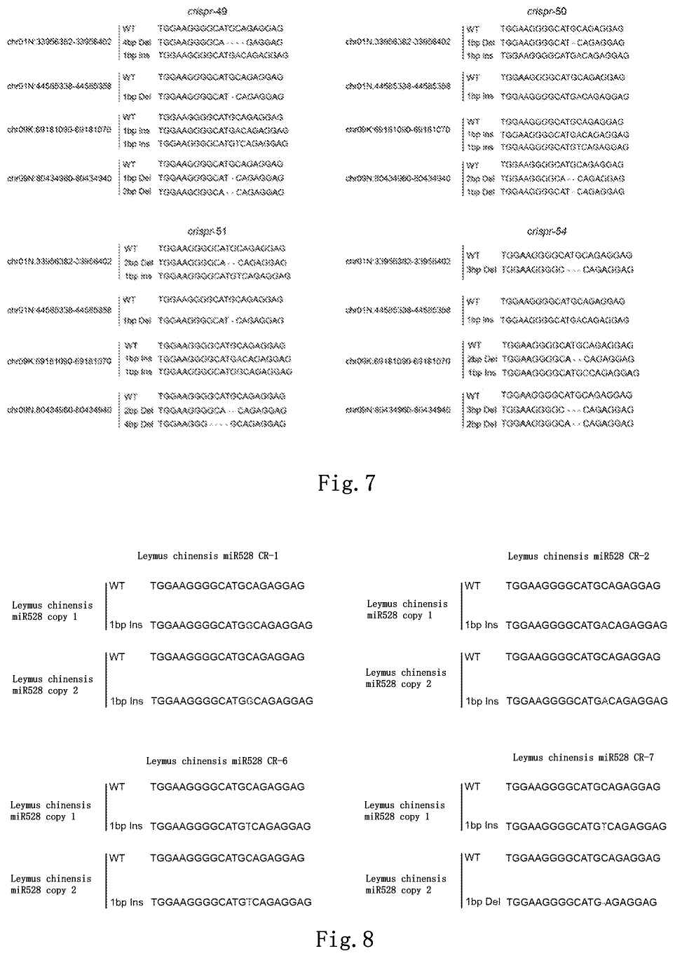

The results are shown in FIG. 7. As compared with the control line transformed with the empty vector, the transgenic line in which miR528 was inhibited exhibited more newly sprouted buds and a greater number of branches. It was demonstrated that the transgenic lines with suppressed miR528 exhibited a stronger regeneration capacity.

### Example 8. Construction of miR528 CRISPR/Cas9 Transgenic Vector

The CRISPR/Cas9 knockout vector pCXUN-Cas9 (see Chinese patent application CN201610639854.3) was generously provided by Professor Wang Rongchen. For the amplification of the guide RNA expression cassette specifically targeting the miR528 coding sequence, the first half part of guide RNA expression cassette was amplified using forward primer U3-F (SEQ ID NO: 14) and reverse primer HX8366 (SEQ ID NO:15), and the latter half part of guide RNA expression cassette was amplified using forward primer HX8365 (SEQ ID NO: 16) and reverse primer U3-R (SEQ ID NO: 17), with OsU3-sgRNA plasmid (see Chinese patent application CN201810059520.8) serving as a template. The two fragments were connected to the Kpnl restriction site of pCXUN-Cas9 through homologous recombination to create a CRISPR/Cas9 vector for targeted editing of the miR528 encoding sequence.

The PCR system in the vector construction process was as follows: 10 ng of plasmid template, 2 μl of forward primer (10 μM), 2 μl of reverse primer (10 μM), 25 μl of 2× Phanta Max Buffer, 1 μl of dNTP Mix (10 mM each), Phanta Max Super-Fidelity DNA Polymerase (1 U/μl) (Nanjing Vazyme Biotech Co., Ltd., P505-d1) 1 μl, and ddH₂O added to a total volume of 50 μl.

The PCR reaction program was as follows: 94° C. for 2 min; (94° C. for 30 s, 58° C. for 30 s, 72° C. for 30 s)×32 cycles; 72° C. for 10 min, and then maintained at 16° C.

The enzyme digestion system was as follows: 5 μl of 10× CutSmart Buffer, 1 μl of restriction endonuclease (Kpnl, 4U/μl), 1 μg of pCXUN-Cas9 plasmid, ddH₂O added to a total volume of 50 μl, and digestion at 37° C. for at least 8 hours.

### Example 9. Gene Identification in miR528 CRISPR/Cas9 Gene-Edited Panicum virgatum and Leymus chinensis

Using the agrobacterium-mediated callus infection system, the miR528 CRISPR/Cas9 vector constructed in Example 8 was transformed into calli of Panicum virgatum and Leymus chinensis. The obtained transgenic regenerated plants were genotyped. Forward primer CP1102 (SEQ ID NO:18) and reverse primer CP1103 (SEQ ID NO:19) were used to identify miR528-copy 1 of Panicum virgatum (chr01N: 33956382-33956402 in FIG. 8); forward primer CF8647 (SEQ ID NO:20) and reverse primer CF8648 (SEQ ID NO:21) were used to identify miR528-copy 2 of Panicum virgatum (chr01N: 44585338-44585358 in FIG. 8); forward primer CF8639 (SEQ ID NO:22) and reverse primer CF8640 (SEQ ID NO:23) were used to identify miR528-copy 3 of Panicum virgatum (chr09K: 69181090-69181070 in FIG. 8); forward primer CF8641 (SEQ ID NO:24) and reverse primer CF8642 (SEQ ID NO: 25) were used to identify miR528-copy 4 of Panicum virgatum (chr09N: 80434960-80434940 in FIG. 8). Forward primer CP4085 (SEQ ID NO:26) and reverse primer CF4086 (SEQ ID NO:27) were used to identify miR528-copy 1 of Leymus chinensis; forward primer CP4089 (SEQ ID NO:28) and reverse primer CF4090 (SEQ ID NO:29) were used to identify miR528-copy 2 of Leymus chinensis. PCR products were sequenced to identify the editing type of each regenerated plant.

The PCR system consisted of: 50 ng of genomic DNA, 0.8 μl of forward primer (10 μM), 0.8 μl of reverse primer (10 μM), 10 μl of 2× Phanta Max Buffer, 0.4 μl of dNTP Mix (10 mM each), 0.4 μl of Phanta Max Super-Fidelity DNA Polymerase (1 Uμl) (Nanjing Vazyme Biotech Co., Ltd., P505-d1), and ddH₂O added to a total volume of 20 μl.

The PCR reaction program was as follows: 94° C. for 2 min; (94° C. for 30 s, 58° C. for 30 s, 72° C. for 30 s)×35 cycles; 72° C. for 10 min, and maintained at 16° C.

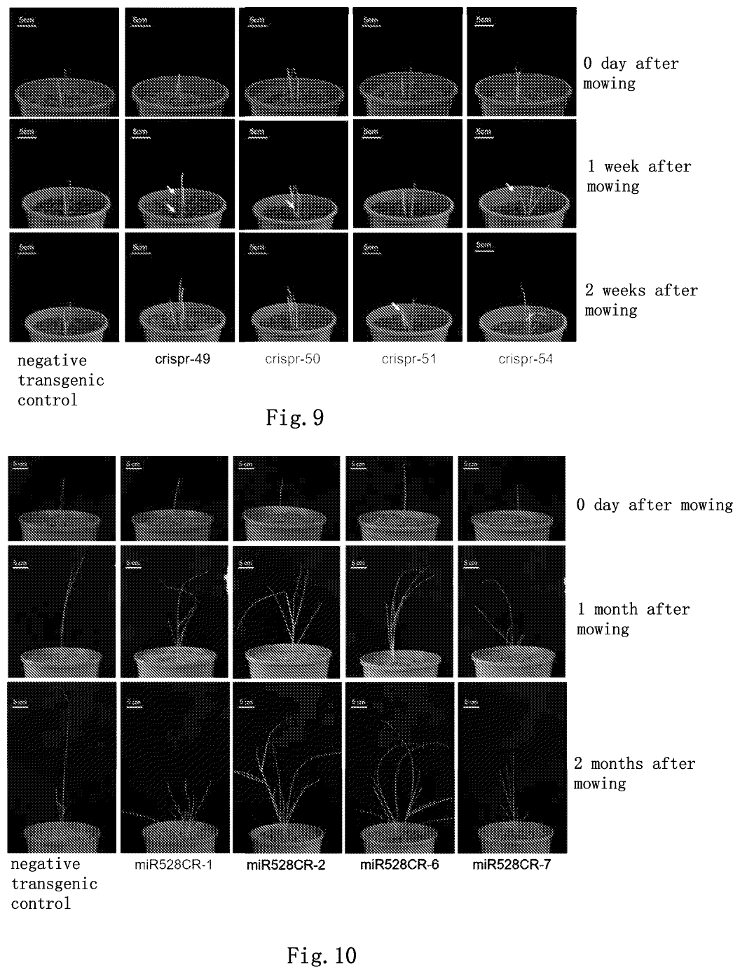

The sequencing results were compared with the reference genome sequence. The detection results of miR528 sequence editing of Panicum virgatum and Leymus chinensis were shown in FIG. 8 and FIG. 9 respectively. Crispr-49, crispr-50, crispr-51, and crispr-54 in FIG. 8 represented distinct transgenic regenerated plants of Panicum virgatum. Leymus chinensis miR528 CR-1, Leymus chinensis miR528 CR-2, Leymus chinensis miR528 CR-6, and Leymus chinensis miR528 CR-7 in FIG. 9 represented distinct transgenic regenerated plants of Leymus chinensis. It could be seen that transgenic regenerated plants of Panicum virgatum and Leymus chinensis with changes in the miR528 sequence were obtained using CRISPR/Cas9 technology.

### Example 10. Identification of Regeneration Capacity of miR528 CRISPR/Cas9 Gene-Edited Panicum virgatum and Leymus chinensis

The top parts of 4-month-old seedlings of miR528-knockout transgenic Panicum virgatum, cultured in vermiculite nutrient soil, were uniformly mowed, leaving only 15 cm-long stems above ground. Each plant was cut into equal sections from the roots, replanted and cultured in the vermiculite nutrient soil, and the phenotype was checked for new tillers after one and two weeks, respectively.

As shown in FIG. 10, the transgenic Panicum virgatum with miR528 knockout exhibited a faster budding rate, indicating that the transgenic Panicum virgatum with miR528 knockout had a stronger regeneration capacity after mowing.

A single tiller of the miR528 knockout transgenic Leymus chinensis, grown in the nutrient soil for 6 months, was kept and transplanted into new vermiculite nutrient soil. The phenotype was checked for new tillers one month and two months later, respectively.

As shown in FIG. 11, the miR528 knockout transgenic Leymus chinensis exhibited a stronger regeneration capacity and a higher number of tillers, indicating that miR528 knockout could enhance regeneration capacity and tillering capacity of Leymus chinensis.

The embodiments described herein are for illustration purpose only (as examples), and various modifications or changes made by skilled individuals should also be considered within the essential scope of the patent application.

### INDUSTRIAL APPLICABILITY

The invention provides the application of miR528 in the production and breeding of gramineous forage grasses. By down-regulating the expression and/or function of miR528 in gramineous forage grasses, the tiller number and regeneration capacity of gramineous forage grasses can be significantly improved, thereby contributing to the production and breeding of gramineous forage grasses, making the invention suitable for industrial applications.

1. A method for enhancing the tillering capacity and/or regeneration capacity of gramineous forage grasses, which comprises down-regulation of the expression and/or function of miR528 in gramineous forage grasses.

2. The method of claim 1, wherein the down-regulation of the expression of miR528 is performed by knockout or gene editing of coding gene, MIR528.

3. The method of claim 2, wherein the gene editing is the editing of a transcribed region of MIR528, and/or its upstream regulatory regions, and/or its downstream regulatory regions.

4. The method of claim 2, wherein the gene editing is performed by a CRISPR/Cas9 system.

5. The method of claim 1, wherein the down-regulation of function of miR528 is performed by expressing an artificial target mimic sequence and/or a short tandem target mimic, that recognizes and interacts with miR528 without being cleaved by miR528, in gramineous forage grasses.

6. The method of claim 5, wherein a vector expressing the artificial target mimic sequence and/or the short tandem target mimic is introduced into the gramineous forage grasses, and wherein the vector expresses the artificial target mimic sequence and/or the short tandem target mimic constitutively.

7. The method of claim 5, wherein the artificial target mimic sequence comprises or consists of the nucleotide sequence shown in SEQ ID NO: 1.

8. The method of claim 5, wherein the short tandem target mimic comprises or consists of the nucleotide sequence shown in SEQ ID NO:3.

9. The method of claim 1, wherein the gramineous forage grasses comprise Panicum virgatum, Leymus chinensis, Brachypodium distachyon, Miscanthus, Elytrigia intermedia, Uraria crinita, Setaria viridis, Anastatica, Elymus sibiricus, Panicum miliaceum and Bromus inermis.

10. A method for breeding of new gramineous forage grass lines with enhanced tillering capacity and/or regeneration capacity, comprising down-regulating the expression and/or function of miR528 in the new gramineous forage grass lines.

11. An artificial target mimic sequence comprising or consisting of the nucleotide sequence shown in SEQ ID NO:1.

12. A short tandem target mimic comprising or consisting of the nucleotide sequence shown in SEQ ID NO:3.
